(19) United States
(12) Patent Application Publication
(10) Pub. No.: US 2010/0144855 A1
(43) Pub. Date: Jun. 10, 2010

(54) APPLICATION OF SEDOISOPROSAN FOR PREPARATION OF MEDICINE FOR TREATMENT OF HUMAN HBeAg POSITIVE CHRONIC HEPATITIS B

(75) Inventors: Qiaolong Zhang, Jieyang City (CN); Guoai Zheng, Jieyang City (CN); Mingfang Zhang, Jieyang City (CN); Manxiang Huang, Jieyang City (CN); Chunxu Zuo, Jieyang City (CN)

Correspondence Address: SCHMEISER, OLSEN & WATTS, 22 CENTURY HILL DRIVE, SUITE 302, LATHAM, NY, 12110, US

(73) Assignee: GUANGDONG YUELONG PHARMACEUTICAL CO., LTD., Jieyang City (CN)

(21) Appl. No.: 12/535,136

(22) Filed: Aug. 4, 2009

(30) Foreign Application Priority Data
Dec. 5, 2008 (CN) 200810219700.4

(51) Int. Cl. A61K 31/35

(52) U.S. Cl. 514/455

(57) ABSTRACT

The present invention disclosed the application of sedoisoprosan for preparation of medicine for therapy on human HBeAg positive chronic hepatitis B. Results of the clinical trials indicated that sedoisoprosan could effectively inhibit hepatitis B virus, the anti-virus effect is enhanced as the dose is increased and the incidence of adverse events is low. Thus, sedoisoprosan is safe and effective for therapy on human HBeAg positive chronic hepatitis B.

### FIELD OF THE INVENTION

The present invention relates generally to the application of sedoisoprosan in pharmaceutical industry, and more specially relates to the application of sedoisoprosan for preparation of medicine for therapy on chronic hepatitis B.

### BACKGROUND OF THE INVENTION

Chinese patent CN96115901.4 has disclosed an anti hepatitis B virus active compound named hylotelephin with the following structure A, the chemical name of which is isopropylidene sedoheptulosan, i.e. sedoisoprosan.

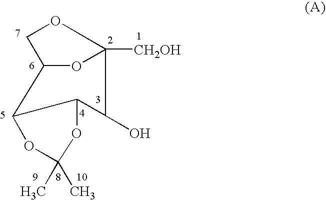

Sedoisoprosan is an active compound produced by condensation of sedoheptulosan and acetone, wherein sedoheptulosan is extracted from hylotelephium spectabile, hylotelephium erythrosticlum or orostachys fimbriatus. Results of the pharmacological study on ducks which were infected with hepatitis B virus indicated that serum DHBV-DNA levels obviously decreased at the seventh and fourteenth day and no toxic reactions or side effect were found after intraperitoneal injection of sedoisoprosan (200 mg/kg or 100 mg/kg) twice a day for 14 days.

Further, the animal experiments revealed the immunomodulatory activity of sedoisoprosan, indicating that sedoisoprosan could enhance the activity of ACP and TNE The vitro experiments indicated that sedoisoprosan associated with ConA and LPS could enhance the mitogenic activity of ConA and LPS, and promote the transformation of lymphocytes. Thus, sedoisoprosan is a biological response modifier with dual immunomodulatory effect.

Although the application of sedoisoprosan for preparation of medicine for anti-duck hepatitis B virus and dual immunomodulation has been disclosed in CN 1054606C, the application of sedoisoprosan for preparation of medicine for therapy on human HBeAg positive chronic hepatitis B remains unclear. Drug safety and efficacy on human needs to be proved by clinical trials, despite the efficacy proved by the cell and animal experiments. The present invention is to define the clinical efficacy, the effective doses and the safety of sedoisoprosan by the clinical study on healthy persons and hepatitis B patients, so as to provide the application of sedoisoprosan for preparation of medicine for therapy on human HBeAg positive chronic hepatitis B.

### SUMMARY OF THE INVENTION

The purpose of the present invention is to provide the application of sedoisoprosan for preparation of medicine for therapy on human HBeAg positive chronic hepatitis B.

The clinical trial protocol was designed according to the randomized, double-blind and controlled principle. The clinical efficacy and the safety of sedoisoprosan for therapy on human HBeAg positive chronic hepatitis B is defined according to the changes of vivo HBV-DNA level, ALT level, vital signs, blood routine indexes, blood biochemical indexes and thyroid function indexes before and after administration and the incidence of adverse events.

Results of the clinical trials indicated that sedoisoprosan could effectively inhibit hepatitis B virus, the anti-virus effect is enhanced as the dose is increased, meaning a dose-effect relationship, and the incidence of adverse events is low. Thus, sedoisoprosan is safe and effective for therapy on human HBeAg positive chronic hepatitis B.

### DETAILED DESCRIPTION OF THE PREFERRED EMBODIMENTS

For better understanding of the essential of the present invention, the application of sedoisoprosan for preparation of medicine for therapy on human HBeAg positive chronic hepatitis B is described by the clinical trials of sedoisoprosan and the results thereof.

### Target Groups

The clinical trials were performed on adult chronic hepatitis B patients who were HBsAg positive, HBeAg positive and HBsAb negative, with HBV-DNA levels more than 10⁵ copies/ml and increased ALT.

Patients enrolled in the clinical trials must meet all the following criteria: signature of informed consent forms; age ≧18 years and ≦65 years; HBsAg positive, HBeAg positive and HBsAb negative for over 6 months; HBV-DNA level ≧10⁵ copies/ml; increase in serum ALT level in 6 months before the first administration (ULN<ALT≦10×ULN for twice, wherein ALT≧2×ULN for once); negative pregnancy tests for women of child-bearing age in 24 hours before the first administration; and effective contraception measures used during the clinical trials and within 3 months after the therapy.

Patients who meet any of the following criteria can not be enrolled: pregnant or lactating women; having received antiviral treatment, interferon treatment, immunomodulatory treatment or cytotoxic treatment for chronic hepatitis B in 6 months before the clinical trials; HAVIgM, HCV-RNA or HCV-Ab, HDV-Ab, HEV-Ab or HTV-Ab positive; creatinine level greater than 1.5 times the normal high limit; suspected liver cancer or alpha-fetoprotein (AFP) level ≧100ng/ml; evidence of esophageal variceal bleeding, ascites or other hepatic decompensation; evidence of fatty liver or early hepatocirrhosis; alcoholism or drug use in the year before the clinical trials; any other serious diseases besides hepatitis which may affect the therapy or evaluation on patients, including heart diseases, pulmonary diseases, nephropathies, digestive diseases, nervous system diseases, psychoses, immune system diseases and cancers; having received other testing drugs in 3 months before the clinical trials; allergies to glycosides or mannite; unable or unwilling to sign informed consent forms; and unable to comply with the orders.

Sixty-four cases were required for the clinical trials.

### Dosage Regimen

Since the effective doses of sedoisoprosan for reducing serum DHBV-DNA levels of ducks infected with hepatitis B virus was 200 mg/kg and 100 mg/kg (intraperitoneal injection, twice a day), doses of sedoisoprosan for the clinical trials may be between 200 mg and 800 mg (intravenous drip, once a day). Results of the tolerance tests indicated that sedoisoprosan of 200 mg, 400 mg, 600 mg and 800 mg in 5% glucose solution (intravenous drip, once a day), was well tolerated. For the purpose of the present invention, three doses of sedoisoprosan, 200 mg, 400 mg and 800 mg in 250ml 5% glucose solution (intravenous drip, within 60 mins), were used in the clinical trials.

Mannite of 400˜800 mg was used for control in the clinical trials, and no adverse events were found in phase I clinical trials.

The half-life of sedoisoprosan is 455.6˜404.5 mins. The animal experiments have indicated that sedoisoprosan could reduce the DHBV-DNA level, inhibit the secretion of HBsAg and HBeAg and regulate the immune system, which is similar to interferon. According to the traditional treatment for chronic hepatitis B with interferon, patients enrolled in the clinical trials were administrated with sedoisoprosan once a day continuously for 4 weeks, then 3 times a week continuously for 8 weeks.

Further, patients enrolled in the clinical trials should not be treated with antiviral drugs, antineoplastic drugs, corticosteroid hormones, immunomodulatory drugs and any other drugs which have anti-HBV effect or enzyme reducing action. Drugs which were used with sedoisoprosan in the clinical trials and reasons for such drug combination must be recorded in case report forms (CRF).

### Grouping Method

Three doses of sedoisoprosan, 200 mg, 400 mg and 800 mg respectively, were used for treatment. Mannite of 800 mg was used for negative control.

Patients enrolled in the clinical trials were randomly divided into four groups, i.e. negative control group (800 mg mannite), low dose group (200 mg sedoisoprosan with 600 mg mannite), median dose group (400 mg sedoisoprosan with 400 mg mannite) and high dose group (800 mg sedoisoprosan) respectively. Each patient was assigned with a CRF No. which was randomly generated by computer.

TABLE 1
Grouping Method and Dosage Regimen

| Groups | Cases | Single Dose | Administration Method |
|---|---|---|---|
| control | 16 | 800 mg mannite | Dissolved in 250 ml 5% |
| group | | | glucose solution, |
| low | 16 | 200 mg sedoisoprosan | intravenous drip within |
| dose group | | with 600 mg mannite | 60 mins, |
| median | 16 | 400 mg sedoisoprosan | once a day for 4 weeks, |
| dose group | | with 400 mg mannite | then 3 times a week for |
| high | 16 | 800 mg sedoisoprosan | 8 weeks. |
| dose group | | | |

### Evaluation Method

Baseline assessment was performed on the day of the first administration, which included medical examination, hematological examination, blood biochemical examination, thyroid function examination, ALT examination and quantitative detection of HBV-DNA. The medical examination included the examination of stature, avoirdupois, blood pressure and pulse rate. The hematological examination included the leucocyte, neutrophil, erythrocyte, platelet and haemoglobin counts, and the prothrombin time. The blood biochemical examination included the examination of serum potassium, sodium, GOT, total bilirubin, AKP, total protein, albumin, total cholesterol, triglyceride, urea nitrogen, creatinine and glucose.

The patients were followed up at the 1st, 2nd, 4th, 8th and 12th week of the treatment. The blood biochemical examination and the quantitative detection of HBV-DNA was performed at the 2nd, 4th, 8th and 12th week of the treatment, and the adverse events were recorded at the same time. The thyroid function examination was performed at the 12th week of the treatment.

### Analysis of Curative Effect

The primary curative effect index was the virologic response rate at the 12th week of the treatment, i.e. the proportion of patients having HBV-DNA levels not more than 2×10⁴ copies/ml or having a decline in HBV-DNA levels of 2 log₁₀ copies/ml compared to the baseline value.

The secondary curative effect indexes included the decrease value of lg(HBV-DNA) after the treatment; the biochemical response rate after the treatment, i.e. the proportion of patients having ALT levels that fell below the normal high limit; the proportion of patients having HBeAg negative conversion and being HBeAb positive after the treatment; and the combined response rate after the treatment, i.e. the proportion of patients having HBV-DNA levels not more than 2×10⁴ copies/ml or having a decline in HBV-DNA levels of 2log₁₀ copies/ml compared to the baseline value, having ALT levels that fell below the normal high limit, having HBeAg negative conversion and being HBeAb positive.

Exact probabilistic method was used for comparison of the virologic response rate, biochemical response rate, virus negative conversion rate and combined response rate between the four groups. Comparison of ALT levels between the four groups was performed by analysis covariance of repeated measures data. Comparison of baseline values between the four groups was performed by χ² test or exact probabilistic test, t test and nonparametric test.

### Analysis of Safety

The safety indexes included vital signs, clinical laboratory parameters, adverse events, important adverse events, serious adverse events and the early termination cases. The safety analysis should be performed on patients who have received at least once administration and were evaluated after the administration.

The detection of vital signs and laboratory parameters and the recording for adverse events was performed at baseline and at the 1st, 2nd, 4th, 8th and 12th week of the treatment. The vital signs and laboratory parameters were evaluated by the changes thereof before and after the treatment. The incidence of adverse events was figured by the proportion of patients having adverse events during the treatment, and the evaluation of adverse events included the kinds, grades and the relationship to the drugs. The adverse events were recorded by descriptive statistics, and comparison of the incidence thereof between the four groups was performed by Fisher probabilistic test.

### Baseline Characteristics

There were 64 cases required for the clinical trials, 16 cases for each group, all of which were required for the baseline assessment.

No significant differences in demographic characteristics, vital signs, blood routine indexes, thyroid function indexes and blood biochemical indexes were found between the four groups at baseline, as shown in table 2 to table 6.

TABLE 2
Demographic Characteristics of The Four Groups

| | | High | Median | Low | Control | | |
|---|---|---|---|---|---|---|---|
| Indexes | | Dose Group | Dose Group | Dose Group | Group | Statistic | P |
| Sex | male | 12 (75.00%) | 13 (81.25%) | 12 (75.00%) | 12 (75.00%) | — | 1.0000 |
| | female | 4 (25.00%) | 3 (18.75%) | 4 (25.00%) | 4 (25.00%) | | |
| Age | | 31.25 ± 8.20 | 27.88 ± 5.43 | 30.81 ± 10.12 | 27.44 ± 6.38 | 1.0303 | 0.3857 |
| Avoirdupois | male | 63.13 ± 10.87 | 63.85 ± 12.31 | 61.00 ± 10.66 | 61.50 ± 10.23 | 0.1814 | 0.9085 |
| (kg) | female | 55.88 ± 2.72 | 52.00 ± 7.94 | 63.63 ± 11.51 | 53.75 ± 5.87 | 1.6690 | 0.2307 |

Comparison of the age and avoirdupois between the four groups was performed by analysis covariance, with F statistics.

TABLE 3
Vital Signs of The Four Groups at Baseline

| | High | Median | Low | Control | | |
|---|---|---|---|---|---|---|
| Indexes | Dose Group | Dose Group | Dose Group | Group | Statistic | P |
| Respiratory | 18.13 ± 1.50 | 19.25 ± 4.14 | 18.31 ± 1.08 | 18.94 ± 1.24 | 0.8041 | 0.4965 |
| frequency | | | | | | |
| (times/min) | | | | | | |
| Systolic Pressure | 115.06 ± 12.70 | 116.19 ± 9.33 | 115.19 ± 6.51 | 114.63 ± 8.07 | 0.0784 | 0.9714 |
| (mmHg) | | | | | | |
| Diastolic Pressure | 75.63 ± 7.48 | 73.94 ± 5.53 | 73.31 ± 6.32 | 74.81 ± 7.98 | 0.3439 | 0.7937 |
| (mmHg) | | | | | | |
| Pulse rate | 74.25 ± 5.71 | 75.19 ± 4.78 | 75.00 ± 6.68 | 74.44 ± 5.86 | 0.0948 | 0.9626 |
| (times/min) | | | | | | |

Comparison of the respiratory frequency, blood pressure and pulse rate between the four groups was performed by analysis covariance, with F statistics.

TABLE 4
Blood Routine Indexes of The Four Groups at Baseline

| | High | Median | Low | Control | | |
|---|---|---|---|---|---|---|
| Indexes | Dose Group | Dose Group | Dose Group | Group | Statistic | P |
| Haemoglobin | 142.69 ± 17.62 | 148.06 ± 19.51 | 141.50 ± 13.64 | 148.19 ± 13.64 | 0.8626 | 0.5300 |
| (g/L) | | | | | | |
| Leucocyte | 5.29 ± 1.12 | 5.22 ± 1.44 | 5.42 ± 1.52 | 5.08 ± 0.94 | 0.4397 | 0.9006 |
| (10⁹/L) | | | | | | |
| Platelet | 149.25 ± 38.70 | 155.00 ± 61.34 | 157.56 ± 67.53 | 143.50 ± 51.86 | 0.4485 | 0.8952 |
| (10⁹/L) | | | | | | |
| Erythrocyte | 4.65 ± 0.60 | 4.78 ± 0.53 | 4.61 ± 0.51 | 4.93 ± 0.63 | 1.0401 | 0.3637 |
| (10¹²/L) | | | | | | |
| Neutrophil | 3.00 ± 1.07 | 2.87 ± 0.96 | 3.09 ± 0.95 | 2.96 ± 0.79 | 0.3916 | 0.9271 |
| (10¹²/L) | | | | | | |
| Lymphocyte | 1.76 ± 0.34 | 1.84 ± 0.77 | 1.69 ± 0.40 | 1.65 ± 0.38 | 0.6535 | 0.7343 |
| (10¹²/L) | | | | | | |

Comparison of the haemoglobin, leucocyte, platelet, erythrocyte, neutrophil and lymphocyte counts between the four groups was performed by analysis covariance, with F statistics.

TABLE 5
Thyroid Function Indexes of The Four Groups at Baseline

| | High | Median | Low | Control | | |
|---|---|---|---|---|---|---|
| Indexes | Dose Group | Dose Group | Dose Group | Group | Statistic | P |
| TSH | 1.53 ± 0.54 | 2.17 ± 1.10 | 1.90 ± 1.19 | 2.04 ± 1.13 | 1.0863 | 0.3251 |
| (mIU/L) | | | | | | |
| Dissociate T4 | 15.67 ± 5.07 | 32.26 ± 42.27 | 13.26 ± 3.25 | 13.48 ± 1.34 | 1.0373 | 0.3807 |
| (ng/L) | | | | | | |
| Total T4 | 95.01 ± 70.92 | 81.16 ± 40.08 | 90.61 ± 50.92 | 105.20 ± 54.99 | 0.4417 | 0.8985 |
| (ng/L) | | | | | | |
| Dissociate T3 | 4.63 ± 0.65 | 4.42 ± 1.10 | 4.96 ± 0.48 | 5.11 ± 0.48 | 1.0715 | 0.3528 |
| (ng/L) | | | | | | |
| Total T3 | 1.89 ± 0.60 | 2.06 ± 0.68 | 1.86 ± 0.95 | 2.28 ± 0.65 | 0.8044 | 0.5902 |
| (ng/L) | | | | | | |

Comparison of the TSH, and the T4 and T3 counts between the four groups was performed by paired t test, with t statisticsnd.

TABLE 6
Blood Biochemical Indexes of The Four Groups at Baseline

| | High | Median | Low | Control |
|---|---|---|---|---|
| Indexes | Dose Group | Dose Group | Dose Group | Group |
| GOT | 83.06 ± 46.23 | 85.31 ± 52.70 | 84.06 ± 36.92 | 90.69 ± 46.16 |
| Total Bilirubin | 16.36 ± 6.04 | 19.69 ± 9.78 | 18.67 ± 8.81 | 15.72 ± 4.74 |
| AKP | 100.00 ± 42.19 | 102.75 ± 33.89 | 105.00 ± 53.33 | 101.44 ± 42.62 |
| Total Protein | 76.61 ± 6.08 | 75.57 ± 5.99 | 75.27 ± 4.81 | 77.12 ± 6.06 |
| Albumin | 44.30 ± 4.00 | 44.26 ± 4.52 | 44.57 ± 3.76 | 45.13 ± 3.30 |
| Total Cholesterol | 4.36 ± 1.24 | 4.24 ± 0.98 | 4.49 ± 0.68 | 3.96 ± 0.48 |
| Triglyceride | 1.11 ± 0.41 | 0.93 ± 0.31 | 1.14 ± 0.43 | 1.03 ± 0.51 |
| Urea Nitrogen | 4.52 ± 1.28 | 4.10 ± 1.21 | 4.57 ± 1.33 | 4.46 ± 1.20 |
| Creatinine | 71.89 ± 16.39 | 74.06 ± 10.60 | 69.10 ± 12.06 | 75.34 ± 16.01 |
| Glucose | 4.89 ± 0.85 | 4.65 ± 0.74 | 4.75 ± 0.70 | 4.66 ± 0.55 |
| Potassium | 3.87 ± 0.30 | 3.86 ± 0.44 | 4.05 ± 0.39 | 4.02 ± 0.50 |
| Sodium | 141.16 ± 3.38 | 141.25 ± 3.12 | 142.26 ± 3.03 | 140.75 ± 2.80 |

When the patients were divided into groups, no significant differences in ALT levels were found between the four groups, but the high dose group and the control group had significant differences in ALT levels at baseline. No liver cirrhosis was found in all groups, but higher abnormal degree of the liver function was found in the control group. There were significant differences in GPT levels between the four groups at baseline, but no significant differences in the other liver function indexes, including HBV-DNA level, prothrombin time and the treatment for hepatitis before grouping, were found, as shown in table 7.

TABLE 7
Liver Function Indexes of The Four Groups at Baseline

| | | High | Median | Low | Control | | |
|---|---|---|---|---|---|---|---|
| Indexes | | Dose Group | Dose Group | Dose Group | Group | Statistic | P |
| Cirrhosis | Yes | 16 | 16 | 16 | 16 | — | — |
| | | (100.00%) | (100.00%) | (100.00%) | (100.00%) | | |
| | No | 0 | 0 | 0 | 0 | — | — |
| | | (0.00%) | (0.00%) | (0.00%) | (0.00%) | | |
| Taking Drugs | No | 8 | 8 | 10 | 10 | — | 0.8400 |
| not for chronic | | (50.00%) | (50.00%) | (62.50%) | (62.50%) | | |
| hepatitis B in | Yes | 8 | 8 | 6 | 6 | — | — |
| Past 6 Months | | (50.00%) | (50.00%) | (37.50%) | (37.50%) | | |
| Taking Drugs | No | 16 | 15 | 15 | 16 | — | 1.0000 |
| for chronic | | (100.00%) | (93.75%) | (93.75%) | (100.00%) | | |
| hepatitis B in | Yes | 0 | 1 | 1 | 0 | — | — |
| Past 6 Months | | (0.00%) | (6.25%) | (6.25%) | (0.00%) | | |

| | | | | | | |
|---|---|---|---|---|---|---|
| ALT Level in The Past | 122.43 ± 53.74 | 163.27 ± 102.19 | 161.85 ± 89.79 | 267.13 ± 273.26 | 5.3084 | 0.1506 |
| (U/L) | | | | | | |
| ALT Level at Baseline | 132.06 ± 74.45 | 141.31 ± 101.94 | 154.81 ± 103.19 | 224.94 ± 120.74 | 7.9863 | 0.0463 |
| (U/L) | | | | | | |
| HBV-DNA Level | 8.95 ± 0.68 | 8.69 ± 0.89 | 8.80 ± 1.38 | 8.35 ± 1.35 | 3.4303 | 0.3299 |
| (10⁵ copies/ml) | | | | | | |
| Prothrombin Time | 13.04 ± 1.03 | 13.40 ± 1.11 | 13.91 ± 1.26 | 13.40 ± 1.22 | 3.6488 | 0.3020 |
| (seconds) | | | | | | |

Comparison of the ALT levels between the four groups was performed by Kruskal-Wallis test, with χ² statistics.

### Curative Effect

There were only 56 cases suitable for the evaluation of curative effect because of the early termination.

No significant differences in virologic response rates were found between the three treatment groups after 12 weeks' treatment, as shown in table 8.

TABLE 8
Comparison of Virologic Response Rates
Between The Four Groups After Treatment

| | | | Virologic | Exact |
|---|---|---|---|---|
| Time | Groups | Cases | Response Rate | Probability Value |
| the 4th | High Dose Group | 16 | 0.00% | 0.1013 |
| week | Median Dose Group | 14 | 0.00% | |
| | Low Dose Group | 13 | 15.38% | |
| | Control Group | 13 | 0.00% | |
| the 8th | High Dose Group | 16 | 12.50% | 0.5994 |
| week | Median Dose Group | 14 | 0.00% | |
| | Low Dose Group | 13 | 15.38% | |
| | Control Group | 13 | 7.69% | |
| the 12th | High Dose Group | 16 | 18.75% | 0.4716 |
| week | Median Dose Group | 14 | 14.29% | |
| | Low Dose Group | 13 | 15.38% | |
| | Control Group | 13 | 0.00% | |

Comparison of the virologic response rates was performed by exact probabilistic test.

There were significant differences in the decrease values of lg(HBV-DNA) within the high dose group after 12 weeks' treatment, but no significant differences were found within the median dose group, the low dose group and the control group. Though no significant differences in the decrease values of lg(HBV-DNA) were found between the four groups after 12 weeks' treatment, the HBV-DNA levels tended to decrease more as the doses increased, presenting a dose-effect relationship, as shown in table 9.

TABLE 9
Comparison of lg(HBV-DNA) levels After 12 Weeks' Treatment

| | The 12th Week | | Comparison | | Comparison | |
|---|---|---|---|---|---|---|
| | Decrease Values | | Within | | Between | |
| | of lg(HBV-DNA) | | A Group | | The Four Groups | |
| | Cases | mean ± SD | Statistic | P | Statistic | P |
| High Dose | 16 | 0.96 ± 1.41 | 61.0 | 0.0006 | 2.9359 | 0.4016 |
| Group | | | | | | |
| Median Dose | 14 | 0.40 ± 0.90 | 17.5 | 0.2439 | | |
| Group | | | | | | |
| Low Dose | 13 | 0.52 ± 1.28 | 14.5 | 0.3396 | | |
| Group | | | | | | |
| Control | 13 | 0.44 ± 0.70 | 25.5 | 0.0803 | | |
| Group | | | | | | |

Comparison within a group was performed by Wilcoxon signed rank rest, with χ² statistics.
Comparison between the four groups was performed by Kruskal-Wallis test, with χ² statistics.

No significant differences in biochemical response rates were found between the four groups after 12 weeks' treatment, and two cases of HBeAg negative conversion were found, one in the high dose group and the other one in the control group, as shown in table 10.

TABLE 10
Comparison of Biochemical Response Rates,
HBeAg Negative Conversion Rates And
Combined Response Rates After 12 Weeks' Treatment

| | High Dose | Median Dose | Low Dose | Control | |
|---|---|---|---|---|---|
| | Group | Group | Group | Group | |
| | (16 cases) | (16 cases) | (16 cases) | (16 cases) | P |
| Biochemical | 18.75% | 12.50% | 6.25% | 12.50% | 0.9532 |
| Response Rates | | | | | |
| HBeAg | 6.25% | 0% | 0% | 6.25% | 1 |
| Negative | | | | | |
| Conversion | | | | | |
| Rates | | | | | |
| Combined | 6.25% | 0% | 0% | 0% | 1 |
| Response | | | | | |
| Rates | | | | | |

Comparison of the biochemical response rates, HBeAg negative conversion rates and combined response rates was performed by exact probabilistic test.

### Safety

All of the 64 cases in the clinical trials were required for the evaluation of safety. Adverse events were found in 12 cases during the clinical trials, two in the high dose group and the control group respectively, four in the median dose group and the low dose group respectively. No central nervous system abnormalities were found in all adverse events, as shown in table 11.

Among the adverse events, important adverse events which were related to the study drug and needed to be treated were found in 3 cases, i.e. CRF16, CRF19 and CRF20 respectively. Besides, serious adverse events were found in one case, i.e. CRF2. At the 4th week of the treatment, the patient with CRF2 had obvious liver dysfunction with progressive increasing ALT level, which was diagnosed as serious chronic hepatitis B. After urgent unblinding, the patient with CRF2 was found to be in the control group, and such serious adverse events were results of untreatment and were unrelated to sedoisoprosan.

TABLE 11
Adverse Events

| CRF No. | Groups | Adverse Events | |
|---|---|---|---|
| | | Liver Dysfunction | |
| 3 | High Dose | Severity | Light |
| | Group | Treatment | Administrated With Liver-protective Drugs |
| | | Dosage adjustment | No |
| | | Relation to the study drug | Unrelated |
| | | Outcome | Remission Without Sequela |
| | | Urinary Tract Infection | |
| 7 | Low Dose | Severity | Light |
| | Group | Treatment | Administrated With Antibiotics |
| | | Dosage adjustment | No |
| | | Relation to the study drug | Unrelated |
| | | Outcome | Remission Without Sequela |
| | | Progressive Increase in ALT Level | |
| 10 | Low Dose | Severity | Middle |
| | Group | Treatment | Observed After Termination of Medication |
| | | Dosage adjustment | — |
| | | Relation to the study drug | Maybe Unrelated |
| | | Outcome | Reexamination of Liver Function After A Week |
| | | a. Alopecia | |
| 14 | Low Dose | Severity | Light |
| | Group | Treatment | Untreated |
| | | Dosage adjustment | No |
| | | Relation to the study drug | Maybe Related |
| | | Outcome | No Change |
| | | b. Gingival Bleeding | |
| | | Severity | Light |
| | | Treatment | Untreated |
| | | Dosage adjustment | No |
| | | Relation to the study drug | Maybe Unrelated |
| | | Outcome | Remission Without Sequela |
| | | c. Pharyngoxerosis | |
| | | Severity | Light |
| | | Treatment | Administrated With Isatis Root Granules |
| | | Dosage adjustment | No |
| | | Relation to the study drug | Unrelated |
| | | Outcome | Remission Without Sequela |
| | | d. Liver Dysfunction | |
| | | Severity | Light |
| | | Treatment | Liver-protective Therapy |
| | | Dosage adjustment | No |
| | | Relation to the study drug | Maybe Unrelated |
| | | Outcome | Remission Without Sequela |
| | | a. Alopecia | |
| 15 | Median Dose | Severity | Light |
| | Group | Treatment | Untreated |
| | | Dosage adjustment | No |
| | | Relation to the study drug | Maybe Related |
| | | Outcome | No Change |
| | | b. Gingival Bleeding | |
| | | Severity | Light |
| | | Treatment | Untreated |
| | | Dosage adjustment | No |
| | | Relation to the study drug | Maybe Unrelated |
| | | Outcome | Remission Without Sequela |
| | | c. Emesis | |
| | | Severity | Light |
| | | Treatment | Untreated |
| | | Dosage adjustment | No |
| | | Relation to the study drug | Maybe Unrelated |
| | | Outcome | Remission Without Sequela |
| | | d. Increase in ALT Level | |
| | | Severity | Light |
| | | Treatment | Administrated With Compound Yiganling |
| | | Dosage adjustment | No |
| | | Relation to the study drug | Maybe Unrelated |
| | | Outcome | Remission Without Sequela |
| | | a. Waist Ache | |
| 16 | Control | Severity | Light |
| | Group | Treatment | Untreated |
| | | Dosage adjustment | No |
| | | Relation to the study drug | Maybe Unrelated |
| | | Outcome | Remission Without Sequela |
| | | b. Alopecia | |
| | | Severity | Light |
| | | Treatment | Untreated |
| | | Dosage adjustment | No |
| | | Relation to the study drug | Maybe Related |
| | | Outcome | No Change |
| | | c. Gingival Bleeding | |
| | | Severity | Light |
| | | Treatment | Gargling With Metronidazole Solution |
| | | Dosage adjustment | No |
| | | Relation to the study drug | Maybe Related |
| | | Outcome | Remission Without Sequela |
| | | d. Hypodynamia | |
| | | Severity | Light |
| | | Treatment | Administrated With Liver-protective Drugs |
| | | Dosage adjustment | No |
| | | Relation to the study drug | Maybe Related |
| | | Outcome | Remission Without Sequela |
| | | Alopecia | |
| 17 | Low Dose | Severity | Light |
| | Group | Treatment | Untreated |
| | | Dosage adjustment | No |
| | | Relation to the study drug | Maybe Related |
| | | Outcome | No Change |
| | | a. Generalized Muscle Ache | |
| 19 | High Dose | Severity | Light |
| | Group | Treatment | Untreated |
| | | Dosage adjustment | Yes |
| | | Relation to the study drug | Maybe Related |
| | | Outcome | Remission Without Sequela |
| | | b. Aggravated Muscle Ache | |
| | | Severity | Middle |
| | | Treatment | Treated By Diammonium Glycyrrihizinate (i.v. |
| | | | drip) |
| | | Dosage adjustment | Yes |
| | | Relation to the study drug | Maybe Related |
| | | Outcome | Remission Without Sequela |
| | | c. Costalgia And Notalgia | |
| | | Severity | Middle |
| | | Treatment | Treated By Diammonium Glycyrrihizinate (i.v. |
| | | | drip) |
| | | Dosage adjustment | Yes |
| | | Relation to the study drug | Maybe Related |
| | | Outcome | Remission Without Sequela |
| | | d. ALT 74 U/L, TBil 133.1 | |
| | | Severity | Middle |
| | | Treatment | Treated By Diammonium Glycyrrihizinate (i.v. |
| | | | drip) |
| | | Dosage adjustment | Yes |
| | | Relation to the study drug | Maybe Related |
| | | Outcome | Remission Without Sequela |
| | | a. Acid Regurgitation | |
| 20 | Median Dose | Severity | Light |
| | Group | Treatment | Treated By Famotidine (20 mg Qd) |
| | | Dosage adjustment | No |
| | | Relation to the study drug | Maybe Related |
| | | Outcome | Remission Without Sequela |
| | | b. Hypodynamia | |
| | | Severity | Middle |
| | | Treatment | Treated By Potenlini (100 ml) |
| | | Dosage adjustment | Yes |
| | | Relation to the study drug | Related |
| | | Outcome | Remission Without Sequela |
| | | c. Epigastric Discomfort | |
| | | Severity | Middle |
| | | Treatment | Treated By Yinzhihuang Injection (31 ml) |
| | | Dosage adjustment | Yes |
| | | Relation to the study drug | Related |
| | | Outcome | Remission Without Sequela |
| | | d. Aggravated Digestive Symptom | |
| | | Severity | Middle |
| | | Treatment | — |
| | | Dosage adjustment | Yes |
| | | Relation to the study drug | Related |
| | | Outcome | Remission Without Sequela |
| | | e. Icterus | |
| | | Severity | Middle |
| | | Treatment | — |
| | | Dosage adjustment | Yes |
| | | Relation to the study drug | Related |
| | | Outcome | Remission Without Sequela |
| | | f. Dark Urine | |
| | | Severity | Middle |
| | | Treatment | — |
| | | Dosage adjustment | Yes |
| | | Relation to the study drug | Related |
| | | Outcome | Remission Without Sequela |
| | | Toothache And Periodontitis | |
| 32 | Median Dose | Severity | Middle |
| | Group | Treatment | Medicated |
| | | Dosage adjustment | No |
| | | Relation to the study drug | Unrelated |
| | | Outcome | Remission Without Sequela |
| | | Upper Respiratory Tract Infection | |
| 60 | Median Dose | Severity | Middle |
| | Group | Treatment | Medicated |
| | | Dosage adjustment | No |
| | | Relation to the study drug | Unrelated |
| | | Outcome | Remission Without Sequela |
| | | a. Abnormal Increase in ALT Level | |
| 64 | Control | Severity | Light |
| | Group | Treatment | Followed up After a Week |
| | | Dosage adjustment | — |
| | | Relation to the study drug | Unrelated |
| | | Outcome | — |

| b. Digestive Symptom | |
|---|---|
| Severity | Middle |
| Treatment | Observed After Termination of Medication |
| Dosage adjustment | — |
| Relation to the study drug | Unrelated |
| Outcome | — |

No significant changes in vital signs, blood routine indexes and blood biochemical indexes, excluding the total bilirubin levels, before and after the treatment were found, as shown in table 12 to table 14. There were significant changes in total bilirubin levels before and after the treatment, but such changes were still within the normal range.

TABLE 12
Changes of Vital Signs Before And After The Treatment

| | | High | Median | Low | Control |
|---|---|---|---|---|---|
| Indexes | Time | Dose Group | Dose Group | Dose Group | Group |
| Avoirdupois | baseline | 63.31 ± 9.93 | 61.63 ± 12.35 | 61.66 ± 10.54 | 59.56 ± 9.78 |
| (kg) | the 1st week | 61.34 ± 9.96 | 62.67 ± 12.11 | 61.72 ± 10.47 | 59.63 ± 9.78 |
| | the 2nd week | 61.41 ± 9.89 | 62.30 ± 12.09 | 61.59 ± 10.80 | 59.88 ± 9.79 |
| | the 4th week | 61.53 ± 9.81 | 61.73 ± 9.86 | 61.57 ± 10.72 | 60.33 ± 9.88 |
| | the 8th week | 62.06 ± 9.20 | 62.70 ± 11.76 | 62.86 ± 11.14 | 60.04 ± 10.69 |
| | the 12th week | 62.67 ± 9.45 | 59.88 ± 8.18 | 63.58 ± 12.09 | 60.77 ± 10.14 |
| Respiratory | baseline | 18.13 ± 1.50 | 19.25 ± 4.14 | 18.31 ± 1.08 | 18.94 ± 1.24 |
| frequency | the 1st week | 18.00 ± 1.86 | 19.13 ± 3.76 | 18.63 ± 1.31 | 18.44 ± 1.46 |
| (times/min) | the 2nd week | 18.38 ± 1.78 | 18.53 ± 3.54 | 18.75 ± 1.57 | 18.50 ± 1.59 |
| | the 4th week | 17.94 ± 1.84 | 18.40 ± 4.03 | 18.60 ± 1.30 | 18.93 ± 1.16 |
| | the 8th week | 18.75 ± 1.24 | 19.27 ± 2.34 | 18.64 ± 1.45 | 19.54 ± 3.28 |
| | the 12th week | 19.13 ± 1.13 | 19.31 ± 2.66 | 19.15 ± 1.28 | 19.38 ± 3.36 |
| Systolic Pressure | baseline | 115.06 ± 12.70 | 116.19 ± 9.33 | 115.19 ± 6.51 | 114.63 ± 8.07 |
| (mmHg) | the 1st week | 115.56 ± 13.17 | 114.33 ± 11.00 | 116.63 ± 8.79 | 114.19 ± 8.07 |
| | the 2nd week | 112.81 ± 12.55 | 114.67 ± 9.85 | 116.00 ± 10.67 | 114.19 ± 9.50 |
| | the 4th week | 112.69 ± 11.69 | 115.80 ± 10.25 | 113.93 ± 9.95 | 117.00 ± 8.43 |
| | the 8th week | 115.19 ± 10.91 | 110.87 ± 7.70 | 115.71 ± 9.75 | 113.15 ± 8.11 |
| | the 12th week | 115.20 ± 11.55 | 115.69 ± 9.67 | 115.85 ± 10.79 | 114.38 ± 10.10 |
| Diastolic Pressure | baseline | 75.63 ± 7.48 | 73.94 ± 5.53 | 73.31 ± 6.32 | 74.81 ± 7.98 |
| (mmHg) | the 1st week | 75.31 ± 7.26 | 71.53 ± 7.53 | 72.38 ± 5.14 | 74.94 ± 6.97 |
| | the 2nd week | 75.31 ± 6.14 | 71.73 ± 6.24 | 73.38 ± 6.01 | 77.13 ± 6.74 |
| | the 4th week | 75.44 ± 6.03 | 73.13 ± 5.69 | 74.87 ± 6.55 | 74.47 ± 6.53 |
| | the 8th week | 77.56 ± 5.05 | 73.73 ± 4.51 | 73.29 ± 5.82 | 74.23 ± 5.67 |
| | the 12th week | 76.73 ± 5.16 | 74.77 ± 5.15 | 74.54 ± 4.94 | 74.69 ± 6.28 |
| Pulse rate | baseline | 74.25 ± 5.71 | 75.19 ± 4.78 | 75.00 ± 6.68 | 74.44 ± 5.86 |
| (times/min) | the 1st week | 76.00 ± 5.81 | 77.60 ± 3.83 | 76.06 ± 5.95 | 73.31 ± 5.20 |
| | the 2nd week | 75.81 ± 5.27 | 75.47 ± 5.36 | 74.13 ± 4.70 | 72.69 ± 5.88 |
| | the 4th week | 76.38 ± 5.95 | 76.40 ± 3.81 | 75.13 ± 4.50 | 74.87 ± 3.50 |
| | the 8th week | 75.75 ± 5.27 | 75.40 ± 3.25 | 73.57 ± 5.65 | 75.77 ± 5.23 |
| | the 12th week | 75.73 ± 4.85 | 78.00 ± 4.53 | 71.54 ± 4.16 | 73.92 ± 4.89 |

TABLE 13
Comparison of Blood Routine Indexes After The Treatment

| | | Comparison | | Comparison | |
|---|---|---|---|---|---|
| | | Within | | Between The | |
| | | A Group | | Four Groups | |
| Indexes | Groups | Statistic | P | Statistic | P |
| | | the 1st week | | | |
| Haemo- | High Dose Group | 0.2319 | 0.8197 | 0.7104 | 0.6806 |
| globin | Median Dose Group | −0.0575 | 0.9550 | | |
| (g/L) | Low Dose Group | −0.2600 | 0.7984 | | |
| | Control Group | 1.2970 | 0.2142 | | |
| Leucocyte | High Dose Group | −1.3866 | 0.1858 | 0.9342 | 0.4605 |
| (10⁹/L) | Median Dose Group | −1.1682 | 0.2622 | | |
| | Low Dose Group | −0.4125 | 0.6858 | | |
| | Control Group | 0.5936 | 0.5616 | | |
| Platelet | High Dose Group | −0.4800 | 0.6382 | 0.6881 | 0.7019 |
| (10⁹/L) | Median Dose Group | 0.0263 | 0.9794 | | |
| | Low Dose Group | −1.4494 | 0.1678 | | |
| | Control Group | 0.3608 | 0.7233 | | |
| Erythrocyte | High Dose Group | 0.6426 | 0.5302 | 0.5292 | 0.8396 |
| (10¹²/L) | Median Dose Group | −0.2909 | 0.7754 | | |
| | Low Dose Group | −0.0588 | 0.9539 | | |
| | Control Group | 0.8382 | 0.4151 | | |
| Neutrophil | High Dose Group | 0.3193 | 0.7539 | 0.9758 | 0.4214 |
| (10¹²/L) | Median Dose Group | −0.8715 | 0.3982 | | |
| | Low Dose Group | 0.0157 | 0.9877 | | |
| | Control Group | 1.6047 | 0.1294 | | |
| Lymphocyte | High Dose Group | −3.0910 | 0.0075 | 1.1497 | 0.2759 |
| (10¹²/L) | Median Dose Group | −0.0584 | 0.9543 | | |
| | Low Dose Group | −0.7671 | 0.4549 | | |
| | Control Group | −0.8644 | 0.4010 | | |
| | | the 2nd week | | | |
| Haemo- | High Dose Group | 0.7339 | 0.4744 | 1.3885 | 0.1349 |
| globin | Median Dose Group | −1.2720 | 0.2241 | | |
| (g/L) | Low Dose Group | −0.6684 | 0.5140 | | |
| | Control Group | 2.2711 | 0.0383 | | |
| Leucocyte | High Dose Group | −0.5908 | 0.5635 | 1.4843 | 0.0972 |
| (10⁹/L) | Median Dose Group | −1.8578 | 0.0843 | | |
| | Low Dose Group | 1.5502 | 0.1419 | | |
| | Control Group | 0.5313 | 0.6030 | | |
| Platelet | High Dose Group | 1.3396 | 0.2003 | 0.7402 | 0.6515 |
| (10⁹/L) | Median Dose Group | −0.4726 | 0.6438 | | |
| | Low Dose Group | −0.1374 | 0.8926 | | |
| | Control Group | 1.1718 | 0.2595 | | |
| Erythrocyte | High Dose Group | 0.7893 | 0.4422 | 1.3763 | 0.1404 |
| (10¹²/L) | Median Dose Group | −1.8923 | 0.0793 | | |
| | Low Dose Group | −0.1608 | 0.8744 | | |
| | Control Group | 1.9195 | 0.0741 | | |
| Neutrophil | High Dose Group | 0.1789 | 0.8605 | 1.2977 | 0.1804 |
| (10¹²/L) | Median Dose Group | −1.1650 | 0.2635 | | |
| | Low Dose Group | 2.2300 | 0.0414 | | |
| | Control Group | 0.8890 | 0.3880 | | |
| Lymphocyte | High Dose Group | −0.8912 | 0.3869 | 0.4960 | 0.8639 |
| (10¹²/L) | Median Dose Group | 0.1652 | 0.8712 | | |
| | Low Dose Group | −0.0104 | 0.9918 | | |
| | Control Group | 0.2746 | 0.7874 | | |
| | | the 4th week | | | |
| Haemo- | High Dose Group | −0.6686 | 0.5139 | 0.3319 | 0.9538 |
| globin | Median Dose Group | −0.5641 | 0.5816 | | |
| (g/L) | Low Dose Group | −0.8136 | 0.4295 | | |
| | Control Group | −0.1384 | 0.8919 | | |
| Leucocyte | High Dose Group | 0.0196 | 0.9846 | 1.2246 | 0.2244 |
| (10⁹/L) | Median Dose Group | −1.6340 | 0.1245 | | |
| | Low Dose Group | 1.1506 | 0.2692 | | |
| | Control Group | −0.0000 | 1.0000 | | |
| Platelet | High Dose Group | 0.7885 | 0.4427 | 0.8308 | 0.5618 |
| (10⁹/L) | Median Dose Group | −1.2165 | 0.2439 | | |
| | Low Dose Group | −0.3043 | 0.7654 | | |
| | Control Group | −0.1975 | 0.8463 | | |
| Erythrocyte | High Dose Group | 0.1087 | 0.9149 | 0.3434 | 0.9492 |
| (10¹²/L) | Median Dose Group | −0.7805 | 0.4481 | | |
| | Low Dose Group | −0.1194 | 0.9067 | | |
| | Control Group | 0.0161 | 0.9874 | | |
| Neutrophil | High Dose Group | 0.0305 | 0.9760 | 0.9983 | 0.4011 |
| (10¹²/L) | Median Dose Group | −1.0629 | 0.3058 | | |
| | Low Dose Group | 1.8582 | 0.0843 | | |
| | Control Group | 0.3353 | 0.7424 | | |
| Lymphocyte | High Dose Group | 0.1009 | 0.9210 | 0.4608 | 0.8874 |
| (10¹²/L) | Median Dose Group | −0.6352 | 0.5355 | | |
| | Low Dose Group | −0.0632 | 0.9505 | | |
| | Control Group | −0.6050 | 0.5549 | | |
| | | the 8th week | | | |
| Haemo- | High Dose Group | −2.7927 | 0.0137 | 0.4269 | 0.9080 |
| globin | Median Dose Group | −1.5758 | 0.1374 | | |
| (g/L) | Low Dose Group | −2.2338 | 0.0453 | | |
| | Control Group | −1.9342 | 0.0770 | | |
| Leucocyte | High Dose Group | −1.5695 | 0.1374 | 1.0449 | 0.3607 |
| (10⁹/L) | Median Dose Group | −1.8271 | 0.0891 | | |
| | Low Dose Group | 0.2369 | 0.8168 | | |
| | Control Group | −0.1705 | 0.8674 | | |
| Platelet | High Dose Group | 1.0754 | 0.2992 | 0.5038 | 0.8582 |
| (10⁹/L) | Median Dose Group | 1.2222 | 0.2418 | | |
| | Low Dose Group | 0.1947 | 0.8489 | | |
| | Control Group | −0.0089 | 0.9931 | | |
| Erythrocyte | High Dose Group | −1.0935 | 0.2914 | 0.5454 | 0.8270 |
| (10¹²/L) | Median Dose Group | −1.7333 | 0.1050 | | |
| | Low Dose Group | −1.1913 | 0.2566 | | |
| | Control Group | −0.6327 | 0.5388 | | |
| Neutrophil | High Dose Group | −1.6167 | 0.1268 | 1.1832 | 0.2530 |
| (10¹²/L) | Median Dose Group | −1.2374 | 0.2363 | | |
| | Low Dose Group | 1.6596 | 0.1229 | | |
| | Control Group | −0.6899 | 0.5034 | | |
| Lymphocyte | High Dose Group | −0.3239 | 0.7505 | 0.8831 | 0.5104 |
| (10¹²/L) | Median Dose Group | −0.9273 | 0.3695 | | |
| | Low Dose Group | −1.1072 | 0.2899 | | |
| | Control Group | 1.1736 | 0.2633 | | |
| | | the 12th week | | | |
| Haemo- | High Dose Group | −3.7282 | 0.0022 | 0.6300 | 0.7558 |
| globin | Median Dose Group | −2.2162 | 0.0468 | | |
| (g/L) | Low Dose Group | −2.4105 | 0.0329 | | |
| | Control Group | −1.8749 | 0.0853 | | |
| Leucocyte | High Dose Group | −1.4506 | 0.1689 | 1.2058 | 0.2384 |
| (10⁹/L) | Median Dose Group | −2.8038 | 0.0159 | | |
| | Low Dose Group | −0.3790 | 0.7113 | | |
| | Control Group | −0.1442 | 0.8878 | | |
| Platelet | High Dose Group | −0.7278 | 0.4788 | 0.6072 | 0.7759 |
| (10⁹/L) | Median Dose Group | 0.5386 | 0.6000 | | |
| | Low Dose Group | −0.1719 | 0.8664 | | |
| | Control Group | −0.8350 | 0.4200 | | |
| Erythrocyte | High Dose Group | −2.2016 | 0.0450 | 0.6676 | 0.7214 |
| (10¹²/L) | Median Dose Group | −1.8858 | 0.0838 | | |
| | Low Dose Group | −0.8777 | 0.3973 | | |
| | Control Group | −0.9751 | 0.3488 | | |
| Neutrophil | High Dose Group | −0.9304 | 0.3679 | 1.2828 | 0.1907 |
| (10¹²/L) | Median Dose Group | −3.1741 | 0.0080 | | |
| | Low Dose Group | 0.1928 | 0.8503 | | |
| | Control Group | −0.0586 | 0.9542 | | |
| Lymphocyte | High Dose Group | −2.2257 | 0.0430 | 1.0060 | 0.3952 |
| (10¹²/L) | Median Dose Group | −1.7756 | 0.1011 | | |
| | Low Dose Group | −0.7085 | 0.4922 | | |
| | Control Group | 0.0285 | 0.9777 | | |

TABLE 14
Comparison of Blood Biochemical Indexes After The Treatment

| | | the 12th week | | | |
|---|---|---|---|---|---|
| | | Comparison | | Comparison | |
| | | Within | | Between The | |
| | | A Group | | Four Groups | |
| Indexes | Groups | Statistic | P | Statistic | P |
| GOT | High Dose Group | −0.2596 | 0.7987 | 0.5003 | 0.8608 |
| | Median Dose Group | 0.1988 | 0.8455 | | |
| | Low Dose Group | 0.7856 | 0.4487 | | |
| | Control Group | 0.7689 | 0.4568 | | |
| Total | High Dose Group | −1.2775 | 0.2208 | 1.7956 | 0.0301 |
| Bilirubin | Median Dose Group | 2.0419 | 0.0620 | | |
| | Low Dose Group | 1.7549 | 0.1070 | | |
| | Control Group | −0.6450 | 0.5310 | | |
| AKP | High Dose Group | 2.2751 | 0.0380 | 0.8103 | 0.5827 |
| | Median Dose Group | 0.5245 | 0.6088 | | |
| | Low Dose Group | 0.4134 | 0.6881 | | |
| | Control Group | 0.0320 | 0.9750 | | |
| Total Protein | High Dose Group | −0.6453 | 0.5285 | 0.2859 | 0.9697 |
| | Median Dose Group | −0.5343 | 0.6022 | | |
| | Low Dose Group | 0.0401 | 0.9688 | | |
| | Control Group | −0.0874 | 0.9318 | | |
| Albumin | High Dose Group | −1.0596 | 0.3061 | 1.0008 | 0.3998 |
| | Median Dose Group | −0.8190 | 0.4276 | | |
| | Low Dose Group | −1.7329 | 0.1110 | | |
| | Control Group | 0.8673 | 0.4028 | | |
| Total | High Dose Group | −0.8436 | 0.4122 | 0.5617 | 0.8141 |
| Cholesterol | Median Dose Group | −0.1128 | 0.8770 | | |
| | Low Dose Group | 0.0496 | 0.2729 | | |
| | Control Group | −1.4109 | 0.2697 | | |
| Triglyceride | High Dose Group | 0.7813 | 0.4468 | 1.0313 | 0.3729 |
| | Median Dose Group | −0.9349 | 0.3669 | | |
| | Low Dose Group | 1.2133 | 0.2529 | | |
| | Control Group | 0.0552 | 0.9569 | | |
| Urea | High Dose Group | −0.9662 | 0.3492 | 1.0120 | 0.3895 |
| Nitrogen | Median Dose Group | −0.0518 | 0.9595 | | |
| | Low Dose Group | −1.8606 | 0.0875 | | |
| | Control Group | −0.3221 | 0.7529 | | |
| Creatinine | High Dose Group | −0.6408 | 0.5313 | 0.3487 | 0.9470 |
| | Median Dose Group | 0.2003 | 0.8443 | | |
| | Low Dose Group | 0.1626 | 0.8736 | | |
| | Control Group | 0.2602 | 0.7991 | | |
| Glucose | High Dose Group | 0.1749 | 0.8637 | 0.5778 | 0.8009 |
| | Median Dose Group | −0.2025 | 0.8432 | | |
| | Low Dose Group | 0.3565 | 0.7289 | | |
| | Control Group | −1.1050 | 0.2927 | | |
| Potassium | High Dose Group | −2.3574 | 0.0324 | 0.6769 | 0.7127 |
| | Median Dose Group | −3.3717 | 0.0050 | | |
| | Low Dose Group | −2.1908 | 0.0489 | | |
| | Control Group | −1.6113 | 0.1331 | | |
| Sodium | High Dose Group | 2.1733 | 0.0462 | 0.6404 | 0.7464 |
| | Median Dose Group | 1.2859 | 0.2209 | | |
| | Low Dose Group | 1.4113 | 0.1836 | | |
| | Control Group | 0.9737 | 0.3494 | | |

### Early Termination Cases

The patient with CRF20 in the median dose group quitted the clinical trials after 8 weeks' treatment because of the abnormal examination indexes (ALT level: 343 U/L, AST level: 517 U/L, TBil level: 68.1 umol/L). The patient with in the low dose group quitted the clinical trials after 6 weeks' treatment because of the progressive increasing ALT level. The patient with CRF2 in the control group quitted the clinical trials after 8 weeks' treatment because of the serious adverse events. No patients in the high dose group quitted the clinical trials for adverse events.

Above results indicated that the highest dose of 800 mg was safe for patients, without any harmful effects on vital signs, blood routine indexes and blood biochemical indexes, etc.

### Compliance

The medicine-taking rate in the patients enrolled in the clinical trials, excluding those who discontinued early, was between 80%˜120%, which presented good compliance, as shown in table 15.

TABLE 15
Analysis of Compliance

| Groups | Cases | Mean | SD | Min | Q1 | Median | Q3 | Max |
|---|---|---|---|---|---|---|---|---|
| High Dose Group | 16 | 208.00 | 0.00 | 208.00 | 208.00 | 208.00 | 208.00 | 208.00 |
| Median Dose Group | 16 | 204.25 | 12.17 | 160.00 | 208.00 | 208.00 | 208.00 | 208.00 |
| Low Dose Group | 16 | 191.00 | 41.61 | 56.00 | 208.00 | 208.00 | 208.00 | 208.00 |
| Control Group | 16 | 175.25 | 61.52 | 28.00 | 160.00 | 208.00 | 208.00 | 208.00 |

### Conclusion

Sedoisoprosan could effectively inhibit hepatitis B virus, the anti-virus effect was enhanced as the dose was increased, meaning a dose-effect relationship, and the incidence of adverse events was low. Thus, sedoisoprosan is safe and effective for therapy on human HBeAg positive chronic hepatitis B.

What is claimed is:

1. Application of sedoisoprosan for preparation of medicine for therapy on human HBeAg positive chronic hepatitis B.
